(19) United States
(12) Patent Application Publication
(10) Pub. No.: US 2026/0013835 A1
(43) Pub. Date: Jan. 15, 2026

(54) METHOD FOR REMOVING ENDOSCOPIC ULTRASOUND RINGING ARTIFACT BASED ON TIME-FREQUENCY ANALYSIS FILTERING

(71) Applicants: HARBIN INSTITUTE OF TECHNOLOGY, WEIHAI, Weihai (CN); HARBIN INSTITUTE OF TECHNOLOGY SUZHOU RESEARCH INSTITUTE, Suzhou (CN)

(72) Inventors: Mingjian SUN, Weihai (CN); Kaicheng YU, Weihai (CN); Dongjian WU, Weihai (CN)

(21) Appl. No.: 19/337,022

(22) Filed: Sep. 23, 2025

Related U.S. Application Data
(63) Continuation of application No. PCT/CN2024/122285, filed on Sep. 29, 2024.

(30) Foreign Application Priority Data
Apr. 26, 2024 (CN) 202410514574.4

(51) Int. Cl. A61B 8/00; A61B 8/12

(52) U.S. Cl. CPC A61B 8/5276; A61B 8/12

(57) ABSTRACT

The invention discloses an adaptive method for removing ringing artifacts in single-element endoscopic ultrasonic based on time-frequency analysis filtering. Steps: obtain original data of endoscopic single-element ultrasonic imaging, perform time-frequency spectrum analysis on adjacent frame signals; determine mutation of the time-frequency spectrum of the signal to be processed relative to reference signal by using an optical flow method; determine a threshold coefficient and spectral distribution factors to set parameters of a time-frequency filter; perform time-frequency filtering on the signal to be processed, and then perform time-domain recovery; calculate index of filtered and original data, and optimize and adjust threshold parameters based on whether index meets standard; perform time-frequency filtering, time-domain reconstruction, and ultrasonic endoscopic image reconstruction. The method nearly eliminates ringing artifacts, restores effective echo signals, and maintains structural integrity during close-range imaging.

### CROSS-REFERENCE TO RELATED APPLICATIONS

This application is a continuation of International Application No. PCT/CN2024/122285, filed on Sep. 29, 2024, which claims priority to Chinese Patent Application No. 202410514574.4, filed on Apr. 26, 2024, both of which are hereby incorporated by reference in their entireties.

### TECHNICAL FIELD

The present invention belongs to the field of interventional ultrasound imaging and relates to a method for removing ringing noise in the field of interventional single-element ultrasound imaging, specifically to an adaptive method for removing ringing artifacts in single-element endoscopic ultrasound based on time-frequency analysis filtering.

### BACKGROUND TECHNOLOGY

Currently, interventional single-element ultrasound imaging is often affected by various artifacts. Taking intravascular ultrasound (IVUS) as an example, the ultrasound ringing artifact is a typical artifact in interventional single-element ultrasound imaging represented by IVUS.

Ringing artifacts are caused by the ultrasonic probe receiving its own transmitted ultrasonic detection signals after being excited by the ultrasonic transceiver. The occurrence of ringing artifacts can result in a series of dense concentric circular bright spots in the central region of the ultrasound image. This makes it impossible for interventional single-element ultrasound probes to effectively acquire tissue structural information when positioned close to the tissue to be imaged, due to the obstruction of ringing artifacts, greatly affecting the overall imaging capability of the system.

Regarding the characteristics of ringing artifacts, there are currently some research conclusions. Due to zero drift generated during the use of ultrasonic transceivers and electromagnetic interference from external devices, ringing artifacts can cause a certain degree of variation in ultrasonic detection signals. When the ultrasonic detection signal reaches the tissue to be imaged and generates an ultrasonic echo signal, a portion of the original detection signal itself will enter the entire ultrasonic signal acquisition process through the ultrasonic transducer. This portion of the ultrasonic detection signal, after coupling, becomes the ultrasonic ringing artifact signal.

Currently, the methods for removing ringing artifacts in the world are relatively limited and cannot effectively distinguish between artifacts and normal echo signals. Due to the specificity and relatively fixed appearance area of ringing artifacts, most current methods involve circular cropping of fixed areas from reconstructed interventional ultrasound images to remove ringing artifacts and enhance the overall image contrast. However, this method is based on image-side processing, ignoring the size of the ringing artifact area and unable to adjust accordingly to changes in external noise and transducer parameters. Furthermore, when the area to be imaged is close to the transducer, causing effective echo signals to be overwhelmed by ringing artifacts, conventional cropping methods will also remove effective imaging information near the transducer. Another method involves filtering the signal in the frequency domain before image reconstruction. This method can only suppress ringing artifacts to a certain extent and also has a suppressive effect on ultrasound echo signals.

The aforementioned conventional processing methods may lead to incorrect reconstruction of the narrower sections close to the catheter in interventional single-element ultrasound endoscopic imaging, such as IVUS, ultimately affecting the acquisition of structural information of the overall luminal wall.

### SUMMARY OF THE INVENTION

To address the problems that interventional single-element ultrasonic endoscopic imaging suffers from ringing artifacts, which severely affect the imaging quality, and existing methods for artifact removal have not achieved satisfactory effects, the present invention provides a method for removing endoscopic ultrasonic ringing artifacts based on time-frequency analysis filtering. This method can almost completely eliminate ringing artifacts, restore effective echo signals, and maintain structural integrity during imaging at close range.

The objective of the present invention is achieved through the following technical solutions:

A method for removing endoscopic ultrasonic ringing artifacts based on time-frequency analysis filtering, comprising the following steps:
step 1: obtain raw data of endoscopic single-element ultrasound imaging, perform time-frequency spectrum analysis on adjacent frame signals in ultrasound data containing ultrasound echo signals, and obtain time-frequency spectrum of the signals;
step 2: determine the mutation of the time-frequency spectrum of the signal to be processed relative to the time-frequency spectrum of the reference signal by using an optical flow method;
step 3: determine a threshold coefficient and spectral distribution factors, and determine parameters of a time-frequency filter by means of the two parameters;
step 4: perform time-frequency filtering on the signal to be processed by using the time-frequency filter obtained from preliminary calculations, and then perform time-domain recovery;
step 5: after filtering out artifacts using time-frequency filtering, perform index calculations on the filtered data and the original data, and optimize and adjust the threshold parameters based on whether index meets standard;
step 6: perform time-frequency filtering, time-domain reconstruction, and ultrasonic endoscopic image reconstruction on the data by using the time-frequency filter parameters that meet the standard, so as to obtain ultrasonic endoscopic images free of ringing artifacts.

An adaptive device for removing ringing artifacts in single-element endoscopic ultrasound based on time-frequency analysis filtering, comprising a memory and a processor, wherein the memory stores a computer program, and the processor is configured to execute the computer program to implement the aforementioned adaptive method for removing ringing artifacts in single-element endoscopic ultrasound based on time-frequency analysis filtering.

Compared to existing technologies, the present invention has the following advantages:

The present invention can precisely and effectively reduce the “ringing” artifact effect in interventional single-element ultrasonic endoscopic imaging, eliminate artifact coverage, enhance the signal-to-noise ratio of near-end imaging, and significantly improve the overall structural imaging quality.

### DESCRIPTION OF DRAWINGS

FIG. 1 shows the composition of the raw signal from a single-element ultrasound;

FIG. 2 is a flowchart of a method for removing endoscopic ultrasound ringing artifact based on time-frequency analysis filtering;

FIG. 3 is the time-frequency spectrum image of the reference signal;

FIG. 4 is the time-frequency spectrum image of the signal to be processed;

FIG. 5 is an image of the changed part in the time-frequency spectrum of the signal to be processed relative to the time-frequency spectrum of the reference signal;

FIG. 6 is a comparison matrix (visualized) used for comparison with the time-frequency spectrum variation section;

FIG. 7 is the time-frequency filter image obtained through preliminary calculation;

FIG. 8 shows the ultrasound echo signal with ultrasonic ringing artifacts and the ultrasound echo signal image after removing the ultrasonic ringing artifacts;

FIG. 9 is an image with ringing artifacts reconstructed by the method of the present invention;

FIG. 10 is the image without ringing artifacts reconstructed by the method of the present invention.

### EMBODIMENTS

The technical solution of the present invention will be further described below with reference to the drawings, but it is not limited thereto. Any modifications or equivalent substitutions to the technical solution of the present invention, without departing from the spirit and scope of the technical solution of the present invention, should be included in the protection scope of the present invention.

In a single-element imaging system, one “transmit-detect” operation typically yields only a “line” of data, which is therefore referred to as an A-Line. In the present invention, the A-Line signal will be used to represent the signal obtained from one “transmit-detect” operation.

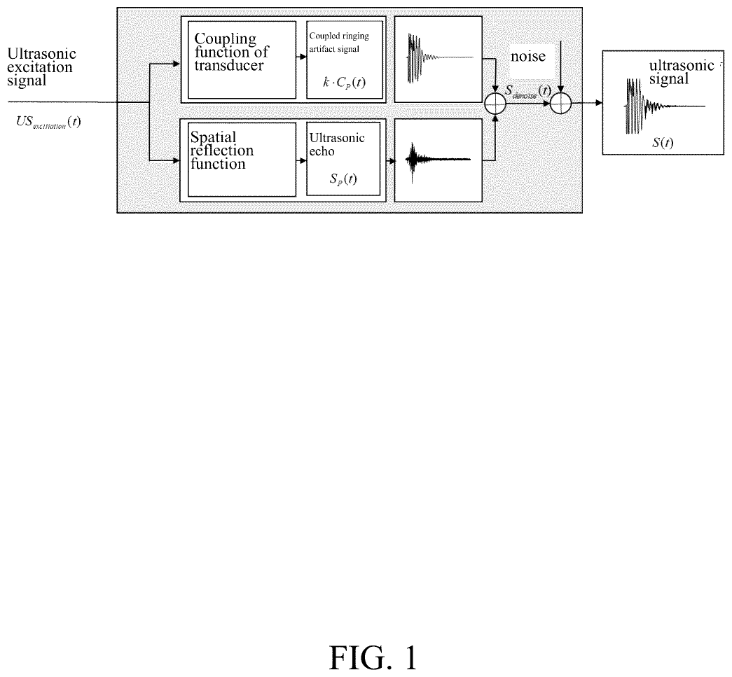

Research on the generation mechanism of the existing “ringing” artifact has revealed that the generated “ringing” artifact follows a certain pattern. The ringing artifacts are produced when the emitted detection ultrasound waves are reversely coupled into the received signal, and their positions is relatively fixed within the entire signal, while their amplitude and shape may vary to some extent due to fluctuations in hardware temperature and parameters. However, there is a strong similarity between adjacent ringing artifacts. The mixing process of the ultrasound echo signal and the ringing artifact signal is shown in FIG. 1. The ultrasound excitation signal US_{exiting}(t), after being coupled by the ultrasound transducer and influenced by temperature and environmental factors, forms the ringing artifact signal k·Cₚ(t), which then mixes with the ultrasound echo signal reflected from the tissue under the ultrasound excitation signal, resulting in the final sampled ultrasound signal S(t)·k is the proportional coefficient of the coupling effect of the ultrasonic transducer as well as temperature and environment.

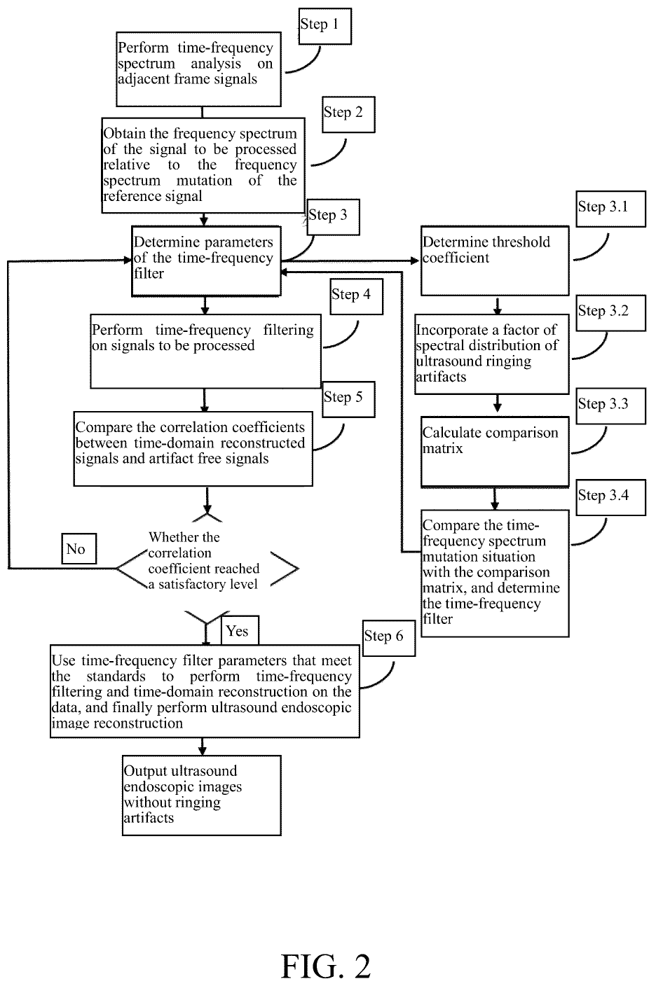

The present invention provides a method for removing endoscopic ultrasound ringing artifact based on time-frequency analysis filtering. The method is based on time-frequency spectrum analysis, and utilizes the strong correlation between ringing artifacts in two adjacent A-line intervals to eliminate the relatively unchanged parts in the time-frequency spectrum, while retaining the changed parts to remove the relatively fixed signal of ringing artifacts. As shown in FIG. 2, the method specifically includes the following steps:

Step 1: Obtain raw data for endoscopic single-element ultrasound imaging, collect ultrasound data containing ultrasound echo signals and the ultrasound data containing only ultrasound ringing artifacts, and perform time-frequency spectrum analysis on signals of two adjacent A-line intervals in the ultrasound data containing ultrasound echo signals. The specific steps are as follows:

Step 11: Collect one frame of B-scan ultrasound data through an ultrasonic endoscopic imaging system. The ultrasound data contains H groups of ultrasound A-line signals, each ultrasound A-line signals consisting of ultrasound ringing artifacts and ultrasound echo signals. The ultrasound data is converted from acoustic signals to electrical signals by an ultrasound transducer and finally collected through a data acquisition card.

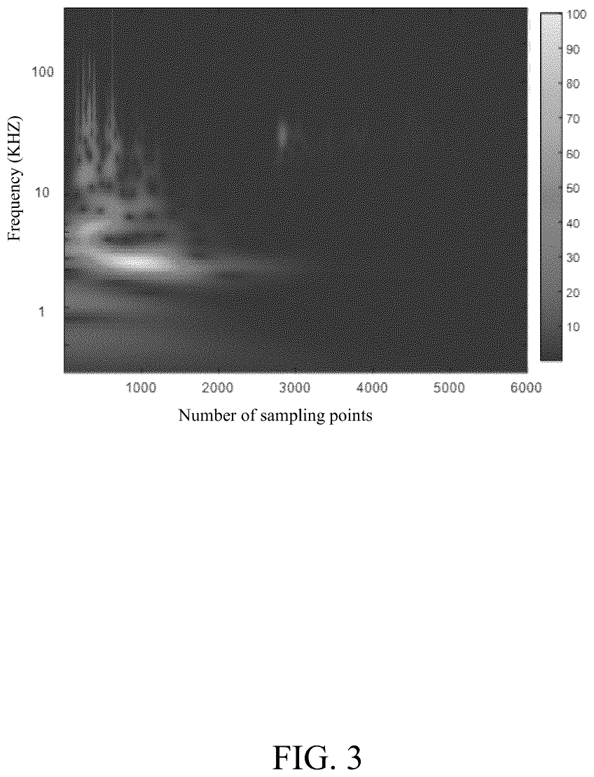

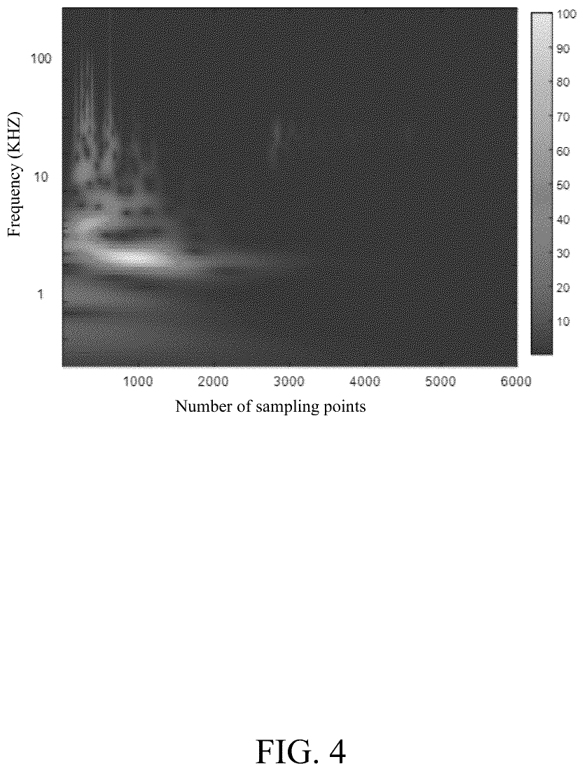

Step 12: Extract signals from two adjacent A-line intervals in the H groups of ultrasound A-line signals, and perform continuous wavelet transform or short-time Fourier transform on the extracted signals to obtain the time-frequency spectra of the two adjacent A-line interval signals, denoted as Sₑ(wₘ, tₙ)_{M×N} and S_{Y}(wₘ, tₙ)_{M×N}, as shown in FIGS. 3 and 4, where wₘ represents the vertical-axis frequency of the time-frequency spectrum, and tₙ represents the number of horizontal-axis sampling points of the time-frequency spectrum. The calculated time-frequency spectrum is a matrix of M×N, where M and N represent the frequency resolution and time resolution of the time-frequency spectrum, respectively.

Step 2: Determine the mutation of the time-frequency spectrum of the signal to be processed relative to the time-frequency spectrum of the reference signal by using an optical flow method. The specific steps are as follows:

Step 21: Based on the time-frequency spectrum of the signal obtained in Step 1, select one frame of time-frequency spectrum as the reference signal Sₑ(wₘ, tₙ)_{M×N};

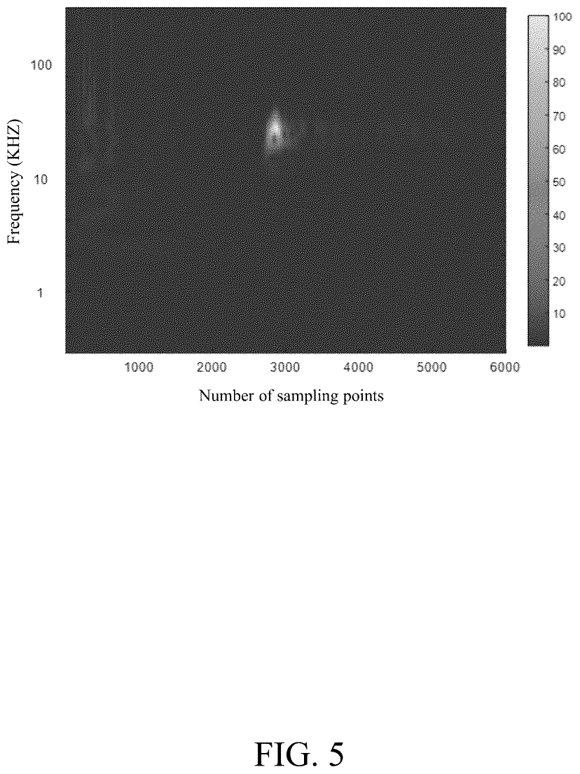

Step 22: Calculate the relatively changing portion of Sᵣ(wₘ, tₙ)_{M×N} with respect to Sₑ(wₘ, tₙ)_{M×N} in the time-frequency spectrum, so as to obtain the moving part D_{M×N} of the time-frequency spectrum of the signal to be processed relative to that of the reference signal. The calculated moving part of the time-frequency spectrum is shown in FIG. 5, and the calculation formula is D_{M×N}=Sₑ(w,tₙ)_{M×N}−Sᵣ(w,tₙ)_{M×N}).

Step 3: Determine a threshold coefficient and spectral distribution factors, and determine parameters of a time-frequency filter by means of the two parameters. The specific steps are as follows:

Step 3.1: Determine a threshold coefficient eff to retain parts of the time-frequency spectrum where the relative change rate is greater than the threshold coefficient, while parts where the relative change rate is less than the threshold coefficient will be eliminated.

Step 3.2: Simultaneously incorporate a factor Γ(wₘ) of spectral distribution of ultrasound ringing artifacts:

$$Γ ( {w}_{m} ) = \vert {\int }_{- ∞}^{+ ∞} {u}_{0} ( t ) {e}^{- jwt} dt \vert$$

Γ(wₘ) is Fourier transform of the ringing artifact u₀(t) itself, which represents the frequency distribution of the ringing artifact. By incorporating this factor, the goal of increasing the probability of eliminating parts with a wider frequency distribution is achieved.

Step 3.3: Multiply the reference time-frequency spectrum Sₑ (wₘ, tₙ)_{M×N} by the threshold coefficient eff, and simultaneously incorporate the factor of spectral distribution Γ(wₘ), to determine the final comparison matrix M_{com}(m, n):

$${M}_{com} ( m , n ) = eff \cdot {S}_{e} ( {w}_{m} , {t}_{n} ) \cdot {e}^{- Γ ( {w}_{m} )} .$$

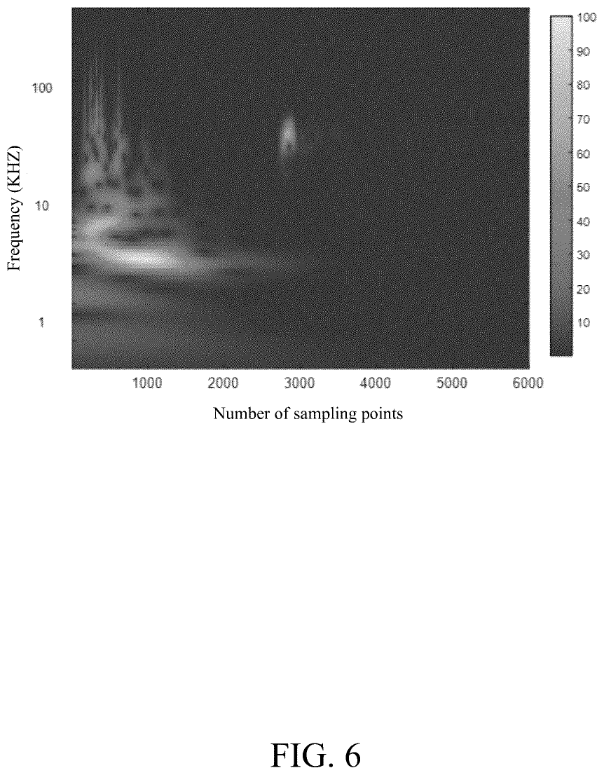

The calculated comparison matrix is shown in FIG. 6.

Step 3.4: Compare the comparison matrix M_{com}(m, n) with moving part of the time-frequency spectrum of the signal to be processed relative to the time-frequency spectrum of the reference signal, set elements in D_{M×N} that are larger than M_{com}(m, n) to 1 and set the elements that are smaller to 0, so as to obtain the preliminary time-frequency filter A(m, n).

$$A ( m , n ) = \{ \begin{matrix}1 & \vert D ( m , n ) \vert > \vert {M}_{com} ( m , n ) \vert \\ 0 & \vert D ( m , n ) \vert \leq \vert {M}_{com} ( m , n ) \vert\end{matrix}$$

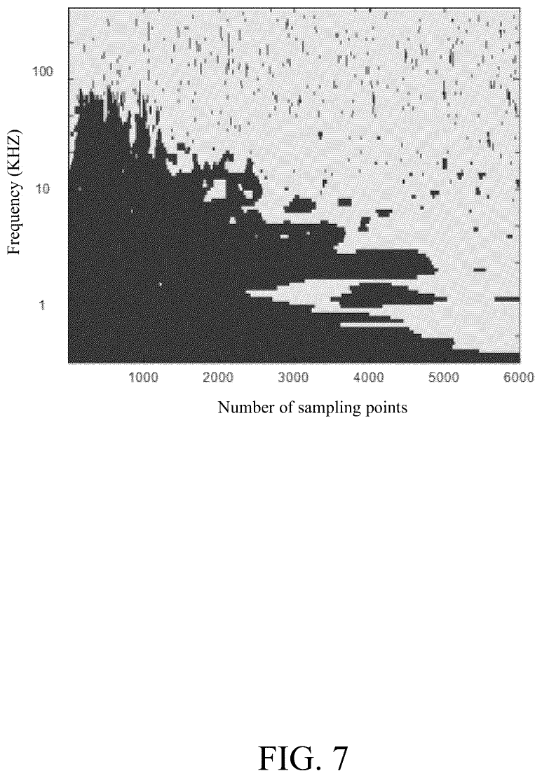

The time-frequency filter image obtained through preliminary calculations is shown in FIG. 7.

Step 4: Perform time-frequency filtering on the signal to be processed by using the time-frequency filter obtained through preliminary calculations, and then perform time-domain recovery. The specific steps are as follows:

Step 41: Perform time-frequency filtering on the time-frequency spectrum of the signal to be processed by using the preliminarily obtained time-frequency filter A(m, n), to obtain the time-frequency spectrum Sᵣₑₛᵤₗₜ(w, tₙ) after filtering:

$${S}_{result} ( {w}_{m} , {t}_{n} ) = A ( m , n ) \cdot {S}_{e} ( {w}_{m} , {t}_{n} )$$

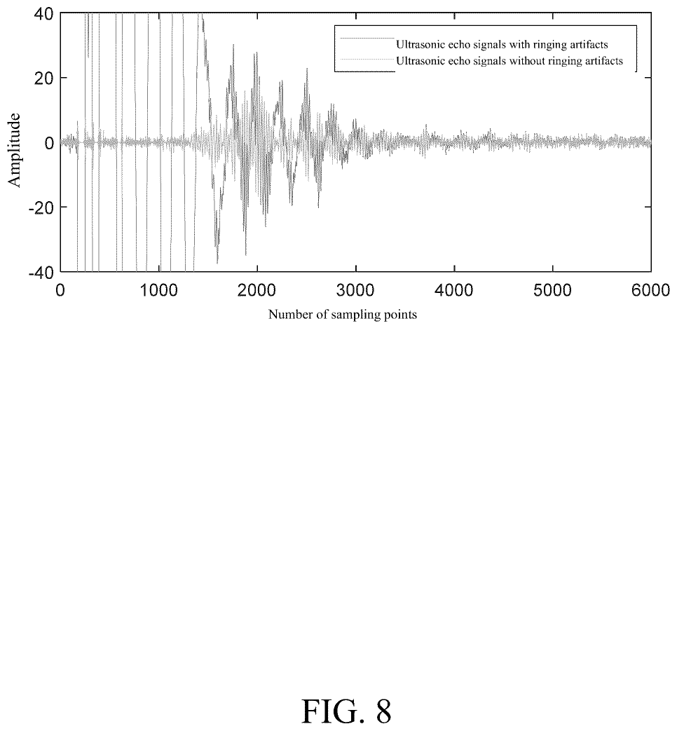

Step 42: Perform time-frequency inverse transform on the time-frequency spectrum Sᵣₑₛᵤₗₜ(w, tₙ) after time-frequency filtering, restore it into a time-domain signal, and obtain an ultrasound echo signal free of ultrasonic ringing artifacts. The restored time-domain signal is shown in FIG. 8.

Step 5: After filtering out artifacts using time-frequency filtering, perform index calculations on the filtered data and the original data, and optimize and adjust the threshold parameters based on whether index meets standard. The specific steps are as follows:

Step 51: Remove the ultrasonic ringing artifact signal from all ultrasonic data containing ultrasonic echo signals, to obtain ultrasonic echo signals u^{clear}ₙᵤₘ(t) without ringing artifacts. This signal is the numth one among all A-line signals, and it is superimposed and mixed with ultrasonic data containing only ultrasonic ringing artifacts to simulate the actual ultrasonic echo signal u^{mix}ₙᵤₘ(t);

Step 52: Repeat the method for removing ultrasonic ringing artifact, to remove the ultrasonic ringing artifacts from the simulated actual ultrasonic echo signals u^{mix}ₙᵤₘ(t), obtaining the ultrasonic echo signals u^{cleared}ₙᵤₘ(t) after artifact removal, calculate the correlation coefficient between the ultrasonic echo signal u^{cleared}ₙᵤₘ(t) and the ultrasonic echo signal u^{clear}ₙᵤₘ(t) without ringing artifacts, repeat the filtering process, and optimize the correlation coefficient between the restored ultrasonic echo signal and the original ultrasonic echo signal by adjusting the value of the threshold coefficient eff.

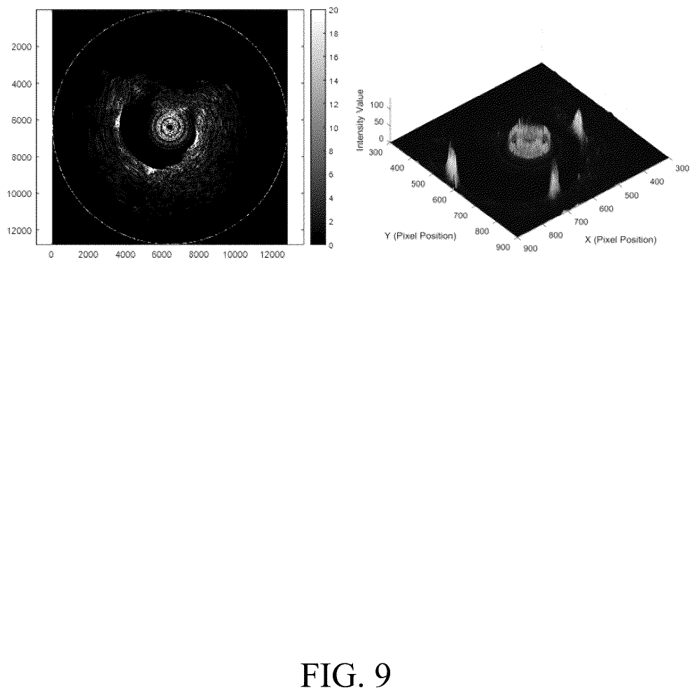

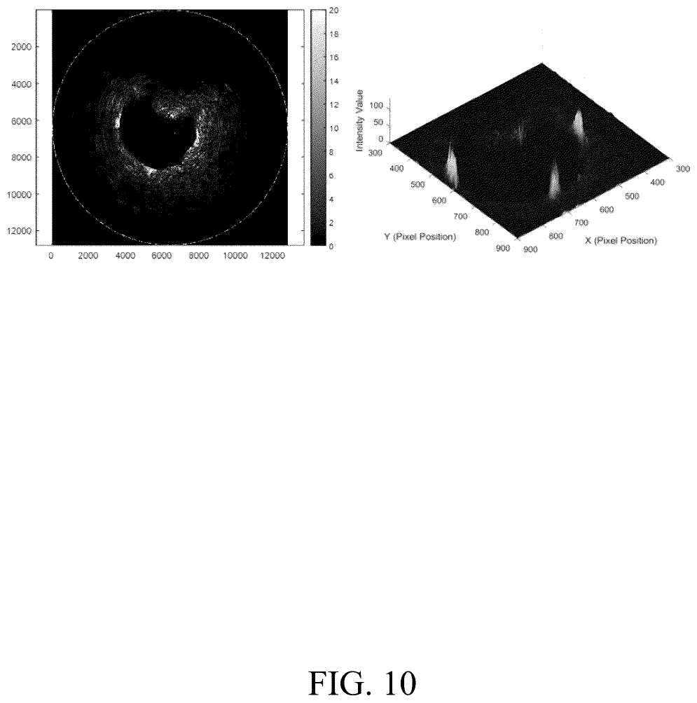

Step 6: Perform time-frequency filtering, time-domain reconstruction, and ultrasonic endoscopic image reconstruction on the data by using the time-frequency filter parameters that meet the criteria, while comparing the effect of artifact removal. The image containing ringing artifacts reconstructed by the method of the present invention is shown in FIG. 9, and the image with ringing artifacts removed and reconstructed by the method of the present invention is shown in FIG. 10. From FIG. 9 and FIG. 10, it can be seen that after removing the ringing artifacts, the image near the center region of the image can be restored and no longer affected by ultrasound ringing artifacts.

The present invention further provides an adaptive device for removing ringing artifacts in single-element endoscopic ultrasonic based on time-frequency analysis filtering. The device comprises a memory and a processor, wherein the memory stores a computer program, and the processor is configured to execute the computer program to implement the aforementioned adaptive method for removing ringing artifacts in single-element endoscopic ultrasonic based on time-frequency analysis filtering.

What is claimed is:

1. An adaptive method for removing ringing artifacts in single-element endoscopic ultrasound based on time-frequency analysis filtering, characterized in that the method comprises following steps:
step 1: obtain raw data of endoscopic single-element ultrasound imaging, perform time-frequency spectrum analysis on adjacent frame signals in ultrasound data containing ultrasound echo signals to obtain time-frequency spectrum of the signals;
step 2: determine a mutation of time-frequency spectrum of the signal to be processed relative to the time-frequency spectrum of reference signal by using an optical flow method;
step 3: determine a threshold coefficient and spectral distribution factors, and determine parameters of a time-frequency filter by means of the two parameters;
step 4: perform time-frequency filtering on signal to be processed by using the time-frequency filter obtained through preliminary calculations, and then perform time-domain recovery;
step 5: after filtering out artifacts using time-frequency filtering, perform index calculations on filtered data and original data, and optimize and adjust threshold parameters based on whether index meets standard;
step 6: perform time-frequency filtering, time-domain reconstruction, and ultrasonic endoscopic image reconstruction on the data by using the parameters of the time-frequency filter that meet the standard, so as to obtain ultrasonic endoscopic images free of ringing artifacts.

2. The adaptive method for removing ringing artifacts in single-element endoscopic ultrasonic based on time-frequency analysis filtering according to claim 1, characterized in that specific steps of step 1 are as follows:
step 11: collect one frame of ultrasound B-scan data through an ultrasonic endoscopic imaging system; the ultrasound data contains H groups of ultrasound A-line signals, each ultrasound A-line signals composing of ultrasound ringing artifacts and ultrasound echo signals;
step 12: extract signals from two adjacent A-line intervals in the H groups of ultrasound A-line signals, and perform continuous wavelet transform or short-time Fourier transform on the extracted signals to obtain the time-frequency spectrum of two adjacent A-line interval signals, denoted as Sₑ (wₘ, tₙ)_{M×N} and Sᵣ(wₘ, tₙ)_{M×N}, respectively, where wₘ represents vertical-axis frequency of the time-frequency spectrum, and tₙ represents number of horizontal-axis sampling points of the time-frequency spectrum; the calculated time-frequency spectrum is a matrix of M×N, where M and N represent frequency resolution and time resolution of the time-frequency spectrum, respectively.

3. The adaptive method for removing ringing artifacts in single-element endoscopic ultrasonic based on time-frequency analysis filtering according to claim 1, characterized in that the specific steps of step 2 are as follows:
step 21: based on the time-frequency spectrum of the signal obtained in Step 1, select one frame of time-frequency spectrum as reference signal Sₑ(wₘ, tₙ)_{M×N};
step 22: calculate relatively changing portion of Sᵣ(wₘ, tₙ)_{M×N} with respect to Sₑ(wₘ, tₙ)_{M×N} in the time-frequency spectrum, to obtain moving part D_{M×N} of the time-frequency spectrum of the signal to be processed relative to the time-frequency spectrum of the reference signal.

4. The adaptive method for removing ringing artifacts in single-element endoscopic ultrasonic based on time-frequency analysis filtering according to claim 3, characterized in that the calculation formula of D_{×N} is as follows:
$${D}_{M \times N} = {{S}_{e} ( w , {t}_{n} )}_{M \times N} - {{S}_{r} ( w , {t}_{n} )}_{M \times N}$$

5. The adaptive method for removing ringing artifacts in single-element endoscopic ultrasonic based on time-frequency analysis filtering according to claim 1, characterized in that specific steps of step 3 are as follows:
step 3.1: determine a threshold coefficient eff to retain parts of the time-frequency spectrum where relative change rate is greater than the threshold coefficient, and which parts where relative change rate is less than the threshold coefficient will be eliminated;
step 3.2: simultaneously incorporate a factor Γ(wₘ) of spectral distribution of ultrasound ringing artifacts:
$$Γ ( {w}_{m} ) = \vert {\int }_{- ∞}^{+ ∞} {u}_{0} ( t ) {e}^{- jwt} dt \vert$$
u₀(t) is a ringing artifact;
step 3.3: multiply reference time-frequency spectrum Sₑ(wₘ, tₙ)_{M×N} by the threshold coefficient eff, and simultaneously incorporate the factor Γ(wₘ) of spectral distribution, to determine final comparison matrix M_{com}(m, n):
$${M}_{com} ( m , n ) = eff \cdot {S}_{e} ( {w}_{m} , {t}_{n} ) \cdot {e}^{- Γ ( {w}_{m} )}$$
step 3.4: compare the comparison matrix M_{com}(m, n) with moving part of the time-frequency spectrum of the signal to be processed relative to the time-frequency spectrum of the reference signal, set elements in D_{M×N} that are larger than M_{com}(m, n) to 1 and set elements that are smaller to 0, to obtain preliminary time-frequency filter A(m, n):
$$A ( m , n ) = \{ \begin{matrix}1 & \vert D ( m , n ) \vert > \vert {M}_{com} ( m , n ) \vert \\ 0 & \vert D ( m , n ) \vert \leq \vert {M}_{com} ( m , n ) \vert\end{matrix} .$$

6. The adaptive method for removing ringing artifacts in single-element endoscopic ultrasonic based on time-frequency analysis filtering according to claim 1, characterized in that specific steps of step 4 are as follows:
step 41: time-frequency filtering on the time-frequency spectrum of the signal to be processed by using the preliminarily obtained time-frequency filter A(m, n), to obtain time-frequency spectrum Sᵣₑₛᵤₗₜ (w,tₙ) after filtering:
$${S}_{result} ( {w}_{m} , {t}_{n} ) = A ( m , n ) \cdot {S}_{e} ( {w}_{m} , {t}_{n} )$$
step 42: perform time-frequency inverse transform on the time-frequency spectrum Sᵣₑₛᵤₗₜ (w, tₙ) after time-frequency filtering, restore it into a time-domain signal, and obtain an ultrasound echo signal free of ultrasonic ringing artifacts.

7. The adaptive method for removing ringing artifacts in single-element endoscopic ultrasonic based on time-frequency analysis filtering according to claim 1, characterized in that specific steps of step 5 are as follows:
step 51: remove the ultrasonic ringing artifact signal from all ultrasonic data containing ultrasonic echo signals, to obtain ultrasonic echo signals u^{clear}ₙᵤₘ(t) without ringing artifacts, the signal is numth one among all A-line signals, and it is superimposed and mixed with ultrasonic data containing only ultrasonic ringing artifacts to simulate actual ultrasonic echo signal u^{mix}ₙᵤₘ(t);
step 52: repeat the method for removing ultrasonic ringing artifact, to remove ultrasonic ringing artifacts from simulated actual ultrasonic echo signals u^{mix}ₙᵤₘ(t), obtaining ultrasonic echo signals u^{cleared}ₙᵤₘ(t) after artifact removal, calculate correlation coefficient between the ultrasonic echo signal u^{cleared}ₙᵤₘ(t) and the ultrasonic echo signal u^{clear}ₙᵤₘ(t) without ringing artifacts, repeat the filtering process, and optimize the correlation coefficient between the restored ultrasonic echo signal and the original ultrasonic echo signal by adjusting the value of the threshold coefficient eff.

8. An adaptive device for removing ringing artifacts in single-element endoscopic ultrasonic based on time-frequency analysis filtering, characterized in that the device comprises a memory and a processor, wherein:
the memory stores computer programs;
the processor is configured to execute the computer program to implement the adaptive method for removing ringing artifacts in single-element endoscopic ultrasonic based on time-frequency analysis filtering according to claim 1.
